(19) United States
(12) Patent Application Publication
(10) Pub. No.: US 2016/0081980 A1
(43) Pub. Date: Mar. 24, 2016

(54) Composition Comprising AMD3100 For Preventing or Treating Bone Diseases

(71) Applicant: KYUNGPOOK NATIONAL UNIVERSITY INDUSTRY-ACADEMIC COOPERATION FOUNDATION, Daegu (KR)

(72) Inventors: Jae Sung Bae, Daegu (KR); Hee Kyung Jin, Daegu (KR)

(21) Appl. No.: 14/889,337

(22) PCT Filed: May 7, 2014
(86) PCT No.: PCT/KR2014/004024

§ 371 Date: Nov. 5, 2015

(30) Foreign Application Priority Data
May 7, 2013 (KR) 10-2013-0051433

(51) Int. Cl. A61K 31/395; A23L 1/30

(52) U.S. Cl. CPC A61K 31/395; A23L 1/30; A23V 2002/00

(57) ABSTRACT

The present invention relates to a composition comprising AMD3100 or a pharmaceutically acceptable salt thereof for preventing or treating bone diseases. AMD3100 according to the present invention can effectively prevent or treat of bone diseases by increasing the SDF-1 level in the blood, inducing mobilization of hematopoietic stem/progenitor cell (HSPC) from the bone to the blood, and reducing the deposition of osteoclast on the bone marrow.

### TECHNICAL FIELD

The present invention relates to a composition for preventing or treating bone diseases, including AMD3100.

### BACKGROUND ART

Bones are active tissues that change constantly over a lifetime. Bones may be classified into cortical bones (compact bones) on the outside surface and trabecular bones (cancellous bones, spongy bones) on the inside surface by the naked eye. Cortical bones have strong physical strength, and play the role of protecting and supporting the physical body, and trabecular bones play the role of resorbing impact or maintaining the change in calcium to a certain level. Even after bones stop growing, old bones break down and are removed (bone resorption), and new bones fill in the empty area (bone formation). Such phenomenon which is repeated over a lifetime is referred to as remodeling of bones. Bone resorption and bone formation should be balanced by balancing interaction between osteoblasts and osteoclasts, so as to maintain homeostasis of bones and maintain the calcium concentration in blood. When blood lacks calcium, in order to supplement this, bone resorption increases to discharge calcium in the bones into the blood, and when bone resorption continues, bones get weak and this causes diseases such as osteoporosis.

Osteoporosis may be classified into postmenopausal osteoporosis where bone resorption increases due to osteoclast activation caused by drastic change in hormones at menopause, and senile osteoporosis where bone formation is reduced by reduction of the function of osteoblast caused by ageing. Fracture caused by osteoporosis results in severe restriction of activity, and hip joint fracture is associated with a high mortality rate of about 15-35%. Accordingly, it is important to diagnose and treat osteoporosis before the occurrence of osteoporotic fracture (Guidelines for Diagnosis and Treatment of Osteoporosis, 2007 and 2008).

The prevalence rate of osteoporosis in Korea has increased by about three times for the past five years as of 2008, and it is reported that the yearly social and economic loss caused by osteoporotic fracture reaches about 1.5 trillion won, which is a very serious level (Guidelines for Diagnosis and Treatment of Osteoporosis, 2007 and 2008). Also, according to the recent national health statistics of 2009, the prevalence rate of osteoporosis in Koreans with the age of 50 or higher and the age of 65 or higher is 23.1% and 42.0%, which is very high among chronic diseases, and thus osteoporosis has become a major problem to national health (National Health Statistics-National Health Nutrition Survey of 2009).

As a conventional therapeutic agent for osteoporosis, bisphosphonate drugs were used. Bisphosphonate is known to be deposited on bone mineral ingredients, and when osteoclasts uptake bones deposited with bisphosphonate, ATP analogue that is not hydrolyzed is formed to present cytotoxicity or reduce activity of osteoclasts in various manners in osteoclasts and cause apoptosis to reduce bone resorption and increase bone density. These drugs are known to be relatively safe. However, when used for a long period of time, they affect the remodeling of bones by normal bone resorption and bone formation or the healing of bones after fracture, thereby deteriorating the elasticity of bones. Thus, there are concerns that they may badly affect bone strength, and in fact, there are reports that the drugs have caused stress fracture in many patients.

Therefore, there is a keen need for discovering a new mechanism for bone metabolism associated with the occurrence of bone diseases and developing a therapeutic agent for preventing or treating bone diseases.

### SUMMARY OF INVENTION

An aspect of the present invention is to provide a composition for preventing or treating bone diseases.

An aspect of the present invention provides a composition for preventing or treating bone diseases, including AMD3100 represented by the following formula 1 or a pharmaceutically acceptable salt thereof:

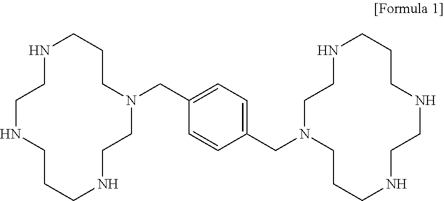 [SMILES: c1cc(CN2CCCNCCNCCCNCC2)ccc1CN1CCCNCCNCCCNCC1]

The composition includes a pharmaceutical composition or food composition.

Bone disease according to the aspect may be osteoporosis, osteomalacia, rickets, fibrous osteitis, aplastic bone disorder or metabolic bone disease, and preferably osteoporosis.

The AMD3100 may release hematopoietic stem cell into blood stream, and reduce osteoclast within bone marrow.

The AMD3100 of the present invention may effectively prevent or treat bone diseases by increasing the SDF-1 level in blood, inducing mobilization of hematopoeitic stem/progenitor cell (HSPC) from bone marrow to blood, and reducing deposition of osteoclast in bone marrow.

### BRIEF DESCRIPTION OF DRAWINGS

FIG. 1 is a view illustrating a timeline of a test designed in the present invention. 12-week-old C57BL/6 mice (n=40) were divided into four groups (Sham/PBS group [n=10]; Sham/AMD3100 group [n=10]; OVX/PBS group [n=10]; OVX/AMD3100 group [n=10]).

FIG. 2 is a view illustrating a steady-state homeostasis fold change in levels of SDF-1 in the supernatant of mouse bone marrow after administration of PBS or AMD3100.

FIG. 3 is a view illustrating a steady-state homeostasis fold change in levels of SDF-1 in the supernatant of mouse plasma after administration of PBS or AMD3100. Data represent mean SEM (ANOVA; Tukeys HSD test. n=4-5 per group). *p<0.05 compared with PBS-treated Sham mice and AMD3100-treated Shame mice or PBS-treated OVX mice and AMD3100-treated OVX mice.

FIG. 4 is the result of analyzing the expression of Osteocalcin, PTHR1, Osterix, and Runx2 from the bone marrow of OVX mice and the control mice by real-time PCR. Data represent mean SEM (ANOVA; Tukeys HSD test. n=4-5 per group). *p<0.05 compared with control or AMD3100-treated mice.

FIG. 5 is a view illustrating the effect of AMD3100 on HSPC mobilization by CFU assay in blood. Data represent mean SEM (ANOVA; Tukeys HSD test. n=4-5 per group). *p<0.05 compared with PBS-treated OVX mice and AMD3100-treated OVX mice.

FIG. 6 is a view illustrating the result of flow cytometry of Lin⁻Sca-1⁺c-kit⁺ cells in the bone marrow of PBS-treated OVX mice and AMD3100-treated OVX mice.

FIG. 7 is a graph illustrating the result of flow cytometry on Lin⁻Sca-1⁺c-kit⁺ cells in the bone marrow of PBS-treated OVX mice and AMD3100-treated OVX mice. Data represent mean SEM (ANOVA; Tukeys HSD test. n=4-5 per group). *p<0.05 compared with PBS-treated OVX mice and AMD3100-treated OVX mice.

FIG. 8 is a view illustrating micro-CT images of the distal femur in each group.

FIG. 9 illustrates the bone mineral density (BMD), bone mineral content (BMC), bone volume fraction (BVF), tissue mineral density (TMD), trabecular number (Tb.N.), trabecular separation (Tb.Sp.), cortical bone mineral density (Cr.BMD), and cortical bone mineral content (Cr.BMC) in the cortical bone and trabecular bone.

FIG. 10 is a view confirming the effect of AMD3100 on the expression of genes associated with osteoclast differentiation by quantitative real-time PCR. Total RNA was extracted from BM cultures treated with or without RANKL in the presence or absence of AMD3100 (25 g/ml) for 3 days. Data represent mean SEM (Anova, Tukeys HSD test. n=3 per group). *P<0.05 compared with AMD3100-treated medium or matched control.

FIG. 11 is a view illustrating the osteoclasts in the trabecular region of OVX mice. Reduction of multi-nucleated TRAP⁺osteoclasts was detected in bone marrow. Arrowheads indicate active TRAP⁺ osteoclasts stained in red (original magnification, ×200).

FIG. 12 is a histogram illustrating the osteoclast number/bone surface [N.Oc/BS (/mm)].

FIG. 13 is a histogram illustrating the osteoblast number/bone surface [N.Ob/BS (/mm)]. Data represent mean SEM (Students t-test. n=4-5 per group). *p<0.05 compared with PBS-treated OVX mice.

### BEST MODE FOR CARRYING OUT THE INVENTION

An aspect of the present invention provides a composition for preventing or treating bone diseases, including AMD3100 represented by the following formula 1 or a pharmaceutically acceptable salt thereof:

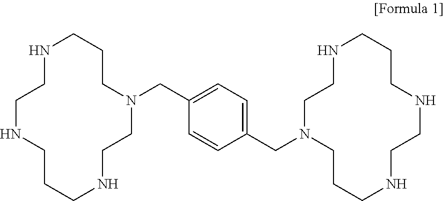 [SMILES: c1cc(CN2CCCNCCNCCCNCC2)ccc1CN1CCCNCCNCCCNCC1]

The composition includes a pharmaceutical composition or food composition. Hereinafter, the present invention is described in detail.

In the present invention, AMD3100 is a new molecular entity of low molecular weight also referred to as Plerixafor, which was first to be authorized as a hematopoietic stem cell mobilizer. The chemical name of the substance is 1,1′-[1,4-Phenylenebis(methylene)]bis[1,4,8,1]-tetraazacyclotetradecanel, its molecular formula is C₂₈H₅₄N₈, and its molecular weight is 502.79 g/mol.

The AMD3100 may be used in the form of a pharmaceutically acceptable salt, and as such salt, adduct salt formed by pharmaceutically acceptable bases may be useful. As such base, inorganic base including alkali metal, or organic base such as amine with strong basicity may be used. Salt formed by using inorganic base may include adduct salt such as sodium salt, potassium salt, calcium salt, magnesium salt, etc., and salt formed by using organic base may include adduct salt such as ethanolamine salt, propanolamine salt, ammonium salt, or general tetraalkylamine salt, etc.

The AMD3100 of the present invention may effectively prevent or treat bone diseases by increasing the SDF-1 level in blood in animal models subjected to OVX experiment, inducing mobilization of hematopoietic stem/progenitor cell (HSPC) from bone marrow to blood, and reducing deposition of osteoclast in bone marrow.

Bone disease according to the aspect may be osteoporosis, osteomalacia, rickets, fibrous osteitis, aplastic bone disorder or metabolic bone disease, and preferably osteoporosis.

When preparing the composition into a pharmaceutical composition for preventing or treating bone diseases, the composition may include a pharmaceutically acceptable carrier. The pharmaceutically acceptable carrier included in the composition may be a carrier generally used for a formulation, and may include lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate and mineral oil, but is not limited thereto. In addition, the pharmaceutical composition may further include a lubricant, a wetting agent, a sweetener, a flavoring agent, an emulsifier, a suspension, a preservative, etc.

The pharmaceutical composition may be administered orally or parenterally.

Parenteral administration may include intravenous injection, subcutaneous injection, intramuscular injection, intraperitoneal injection, endothelial administration, topical administration, intranasal administration, intrapulmonary administration and rectal administration, etc. Because a protein or peptide is digested when administered orally, it is preferred that a composition for oral administration is formulated to coat an active substance or to be protected against degradation in stomach. Also, the pharmaceutical composition may be administered by any device which may transport active substances to target cells.

Proper dose of the pharmaceutical composition may vary according to various factors such as formulating method, administration method, age, weight, gender, pathological state of patient, food, administration time, administration route, excretion rate and reaction sensitivity.

Preferably, a proper dose of the composition is within the range of 0.001 and 100 mg/kg. The term “pharmaceutically effective dose” as used herein refers to an amount sufficient to prevent or treat bone diseases, and preferably osteoporosis.

The composition may be formulated with pharmaceutically acceptable carriers and/or excipients according to a method that may be easily carried out by those skilled in the art, and may be provided in a unit-dose form or enclosed in a multiple-dose vial. Here, the formulation may be in the form of a solution, a suspension, syrup or an emulsion in oily or aqueous medium, or may be extracts, powders, granules, tablets or capsules, and may further include a dispersion agent or a stabilizer. Also, the composition may be administered individually or in combination with other therapeutic agents, and may be administered sequentially or simultaneously with conventional therapeutic agents.

The AMD3100 of the present invention may be added to food or beverage for preventing or improving bone diseases. In this case, the amount of the compound added to the food or beverage may be 0.01 to 15 wt % with respect to the total weight of the food. For health beverage compositions, the compound may be added in a ratio of 0.02 to 5 g based on 100 ml, and preferably in a ratio of 0.3 to 1 g, but this may be easily determined by those skilled in the art according to the product.

The food composition may further include a food supplement additive, which is food-acceptable in addition to the AMD3100, and may be prepared in the form of tablets, capsules, pills, liquids, etc.

More specifically, apart from including the AMD3100, the food composition of the present invention may include without limitation other ingredients as an essential ingredient, and it may include additional ingredients such as various flavoring agents or natural carbohydrates, etc. like other common beverages. Examples of natural carbohydrates include common sugars, including monosaccharides such as glucose, fructose, etc., disaccharides such as maltose, sucrose, etc., and polysaccharides such as dextrin, cyclodextrin, etc., and sugar alcohols such as xylitol, sorbitol, erythritol, etc. In addition, other flavoring agents may be advantageously used, including natural flavoring agents (thaumatin, stevia extract, such as rebaudioside A, glycyrrhizin, etc.), and synthetic flavoring agents (saccharin, aspartame). The ratio of natural carbohydrate is generally about 1 to 20 g, preferably about 5 to 12 g, per 100 ml of the composition of the present invention.

In addition to the above, the food composition of the present invention may contain various nutrients, vitamins, minerals (electrolytes), flavoring agents such as synthetic flavoring agents and natural flavoring agents, coloring agents and improving agents (cheese, chocolate, etc.), pectic acid and salts thereof, alginic acid and salts thereof, organic acids, protective colloidal thickening agents, pH controlling agents, stabilizers, preservatives, glycerin, alcohol, carbonizing agents as used in carbonated beverages, etc. Moreover, the food composition of the present invention may include pulp for the production of natural fruit juice, fruit juice beverage and vegetable beverage. These ingredients may be used independently or in combination with other ingredients. The ratio of the additive is generally selected from a range of 0 to 20 parts by weight with respect to 100 parts by weight of the composition of the present invention.

Hereinafter, the present invention will be described in detail with reference to Examples. However, these examples are for illustrative purposes only, and the present invention is not intended to be limited by the following Examples.

### EXAMPLE 1

### Animals and Treatments

Mice tests were approved by the Kyungpook National University Institutional Animal Care and Use Committee (IACUC). 12-week-old female C57BL/6 mice were purchased from Jackson Laboratory (Bar Harbor, ME). Mice were housed in an air-conditioned room with a 12 hour light/dark cycle at a temperature of 22±2° C. and humidity of 45-65%, and given free access to food and tap water. Mice underwent either sham surgery or ovariectomy (OVX) at 12 weeks of age and were sacrificed at 16 weeks of age. One week after surgery, sham-operated and OVX mice received an intraperitoneal injection with AMD3100 (Sigma-Aldrich #A5602, St. Louis, Mo., sigma Aldrich.com) (5 mg/kg/day) or PBS for 21 days. At the end of treatment, the mice were sacrificed, and blood samples were collected by cardiac puncture for the colony-forming unit (CFU) assay. Femora were removed, fixed with 4% paraformaldehyde in phosphate-buffered physiological saline solution (pH 7.4) for 16 hours, and then stored at 4° C. in 80% ethanol for measurement of bone density.

### EXAMPLE 2

### Quantitative Real-Time PCR

RNA samples were extracted from whole bone marrow (BM) cells of four individual animals per group and isolated from the cultured cells using the RNeasy Mini kit (Qiagen, Hilden, Germany), and the concentration was determined using a Nanodrop ND-1000 spectrophotometer. A total of 5 mg of each RNA was converted to cDNA using the sprint RT complete-oligo (dT) 18 (Clontech, MountainView, Calif., www.clontech.com) according to the manufacturer's guide. The cDNA was quantified using the QuantiTect SYBR Green PCR Kit (Qiagen). The following reaction components were added to each investigated transcript to the indicated end-concentration: forward primer (5 pM), reverse primer (5 pM), and QuantiTect SYBR Green PCR Master mix. The 10 μl master-mix was added to a 0.1 ml tube, and 5 μl volume, containing 100 ng reverse transcribed total RNA, was added as polymerase chain reaction (PCR) template. The tubes were closed, centrifuged, and placed into the Corbett research RG-6000 real-time PCR machine (Corbett LifeScience, Sydney, Australia). The following primers were used:

Osteocalcin
(forward 5′-GGGCAATAAGGTAGTGAACAG-3′,
reverse5′-GCAGCACAGGTCCTA AATAGT-3′),
Osterix
(forward 5′-GCGTATGGCTTCTTTGTGCCT-3′,
reverse5′-AGCTCACTATGGCTCCAGTCC-3′),
Runt-related transcription factor 2 (RUNX2)
(forward 5′-ATACTGGGATGAGGAATGCG-3′,
reverse 5′-CCAAGAAGGCACAGACAGAA-3′),
parathyroid receptor-1 (PTHR1)
(forward: 5′-GGATGATCCACTTCTTGTGC-3′,
reverse 5′-GATTCTGGTGGAGGGACTGT-3′).
Atp6v0d2
(forward 5′-CGGAAAAGAACTCGTGAAGA-3′
reverse 5′-CTGGAAGCCCAGTAAACAGA-3′),
NFATc-1
(forward 5′-AGGTGACACTAGGGGACACA-3′,
reverse 5′-AGTCCCTTCCAAGTTTCCAC-3′),
TRAP
(forward 5′-ACTTCCCCAGCCCTTACTAC-3′),
reverse 5′-TCAGCACATAGCCCACACCG-3′).

### EXAMPLE 3

### Enzyme-Linked Immunosorbent (ELISA) Assay

Murine plasma was collected by cardiac puncture in tubes (a 1 ml syringe containing 50 μl of 100 mM EDTA), and bone marrow was flushed with PBS. After centrifugation, plasma and bone marrow supernatants were collected, and used for detection of SDF-1 protein by ELISA (R&D Systems, Minneapolis, Minn., USA).

### EXAMPLE 4

### Hematopoietic Colony-Forming cell (CFU) Assay

Single-cell suspensions of peripheral blood (PB) after ammonium chloride lysis were plated into 35 mm dishes (3×10⁵ cells/plate) with MethoCult GF M3434 (StemCell Technologies). Hematopoietic colonies were counted and scored after incubation for 12-14 days at 37° C., 5% CO₂, as instructed by the manufacturer.

### EXAMPLE 5

### Flow Cytometry

Bone marrow cells from the femurs and tibias were collected by flushing with 20 ml PBS passed through a 25-gauge needle. After centrifugation at 1,300 rpm for 5 minutes, the supernatant was removed and the cells were then washed by ammonium chloride lysis. Cells were incubated first using a Lineage Cell Depletion Kit magnetic labeling system with the biotinylated lineage antibody cocktail (CD5, CD45R [B220], CD11b, Gr-1 [Ly-6G/C], and Ter-119) for 10 minutes at 4 ° C. and anti-biotin MicroBeads (Milt-enyi Biotec) for an additional 20 minutes at 4° C. Positive immunoselection was performed in a flow cytometer with PE/Cy7-conjugated anti-Sca-1 (BD Pharmingen), APC-conjugated anti-mouse CD117 (c-Kit) (BD Pharmingen), and a FACS Aria (BD Biosciences) using a flow cytometer.

### EXAMPLE 6

### Microcomputed Tomography

For microcomputed tomography (μCT) in vivo imaging, each group of mice was scanned at 8 μm resolution using the eXplore Locus scanner (GE Healthcare). In the femora, scanning regions were confined to the distal metaphysis, extending proximally 1.7 mm from the proximal tip of the primary spongiosa. BMD, BMC, BVF, TMD, Tb.N., Tb.Sp.,

Cr.BMD and Cr.BMC were applied for performance of quantitative analysis using software provided with 2.0+ABA Micro-view of the micro-CT system.

### EXAMPLE 7

### Osteoclast Differentiation

Bone marrow cells were obtained from the femurs and tibias of seven-week-old mice.

The bone marrow suspension was added to plates along with macrophage colony stimulating factor (M-CSF; 30 ng/ml). After culture for 24 hours, the non-adherent cells were collected and resuspended in α-MEM containing 10% FBS. For the osteoclastogenesis experiments, BM-derived macrophages were placed into 6-well plates at a density of 2×10⁶ cells/well in α-MEM with 10% FBS, receptor activator for nuclear factor κB ligand (RANKL; 100 ng/ml) and M-CSF (30 ng/ml) in the presence or absence of AMD3100 (25 mg/ml) for 3 days.

### EXAMPLE 8

### Histological Analysis

For TRAP staining, femurs were fixed in 4% paraformaldehyde for 24 hours, the tissues were then decalcified in 10% EDTA for one week, dehydrated in ethanol, embedded in paraffin, sectioned to 4-μm thickness and stained with hematoxylin and eosin (H&E). For TRAP staining, sections were stained with 225 μM Naphthol AS-MX phosphate (Sigma-Aldrich, St. Louis, Mo., USA), 0.84% N, N-dimethylformamide (Sigma-Aldrich), and 1.33 mM Fast Red Violet LB Salt (Sigma-Aldrich) in 50 mM sodium acetate (pH 5.0) containing 50 mM sodium tartrate, and incubated for 30 minutes. After incubation, sections were washed in distilled water and counterstained with 1% methyl green. Histomorphometric analysis was performed using the Bioquant OSTEOII Program (BIOQUANT Image Analysis Corporation, Nashville, Tenn., USA).

### EXAMPLE9

### Statistical Analysis

The Student's t-test was used for comparison of two groups, whereas Tukey's HSD test and Repeated Measures Analysis of Variance test were used for multi group comparisons according to the SAS statistical package (Release 9.1; SAS Institute Inc., Cary, N.C.). p<0.05 was considered significant.

### EXAMPLE 10

### Confirming SDF-1 Level Increase in the Blood of OVX Mice by AMD3100

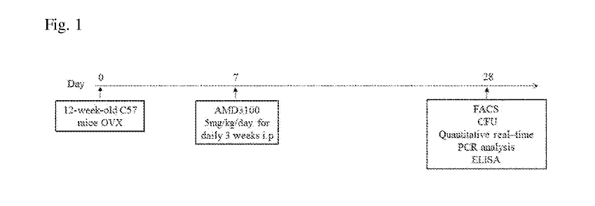

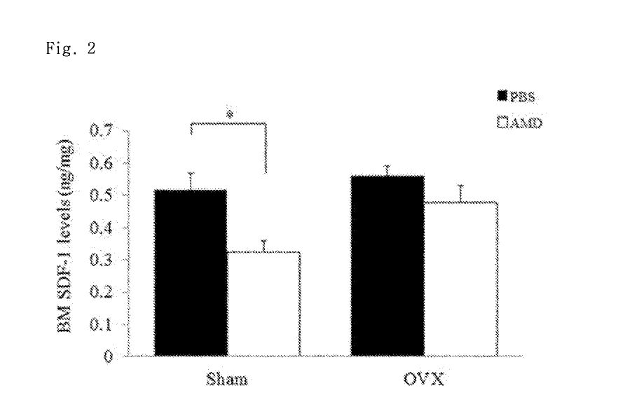

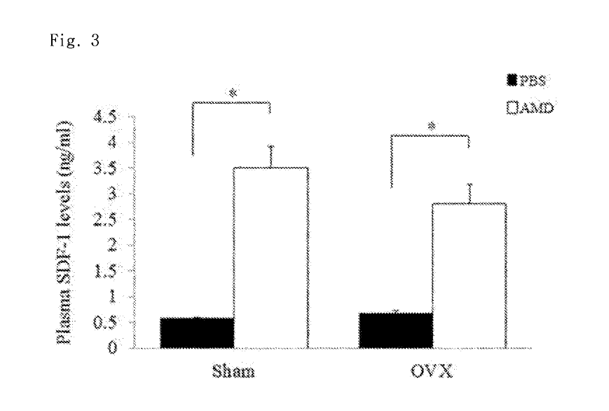

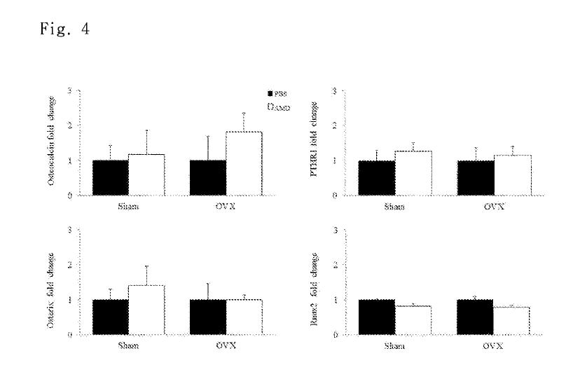

In order to confirm whether AMD3100 has an effect on OVX mice, AMD3100 was injected into OVX mice. The injection protocol is described in FIG. 1. 12-week-old C57BL/6 mice (n=40) were divided into four groups (Sham/PBS group [n=10]; Sham/AMD3100 group [n=10]; OVX/PBS group [n=10]; OVX/AMD3100 group [n=10]). Accordingly, an experiment was performed in order to determine whether treatment with AMD3100 could induce release of SDF-1 and whether it is associated with recruitment of HSPCs. Chemokine stromal cell-derived factor-1 (SDF-1) is also termed CXCL12, and is the most powerful chemoattractant of both human and murine HSPCs. FIG. 2 is a view illustrating a steady-state homeostasis fold change in levels of SDF-1 in the supernatant of mouse bone marrow after administration of PBS or AMD3100. FIG. 3 is a view illustrating a steady-state homeostasis fold change in levels of SDF-1 in the supernatant of mouse plasma after administration of PBS or AMD3100. As illustrated in FIGS. 2 & 3, the levels of functional SDF-1 decreased in bone marrow and increased in plasma of the AMD3100-treated group. These results indicate that treatment with AMD3100 resulted in an increase in SDF-1 in blood and might affect HSPCs mobilization from bone marrow to blood. Next, osteoblast lineage-specific genes, including Osteocalcin, PTHR1, Osterix, and Runx2 were investigated in order to determine whether AMD3100 has an effect on osteoblast activity. However, no significant differences in the levels of osteoblast lineage-specific genes were observed between the groups (FIG. 4). These results confirmed that AMD3100 induced an increase in levels of SDF-1 in blood and did not alter osteoblasts of OVX mice.

### EXAMPLE 11

### Confirming Induction of HSPC Mobilization in OVX Mice by AMD3100

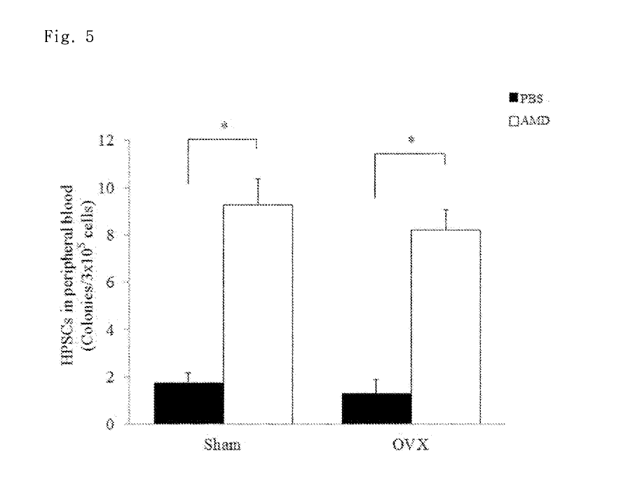

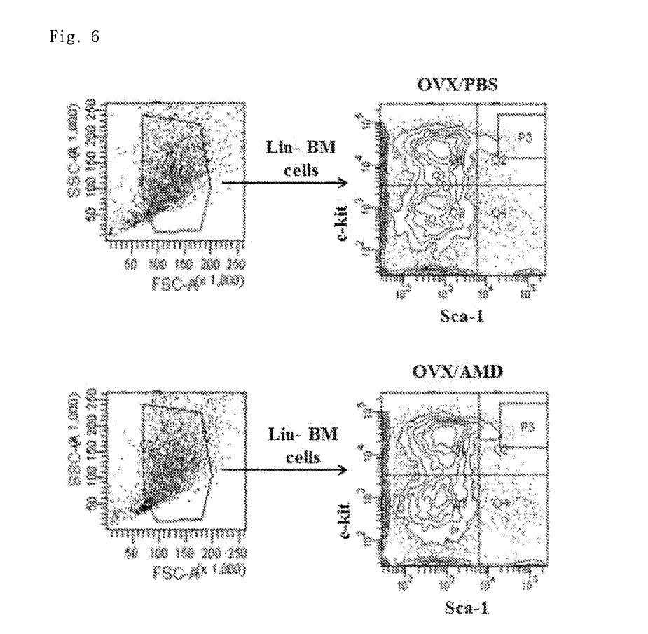

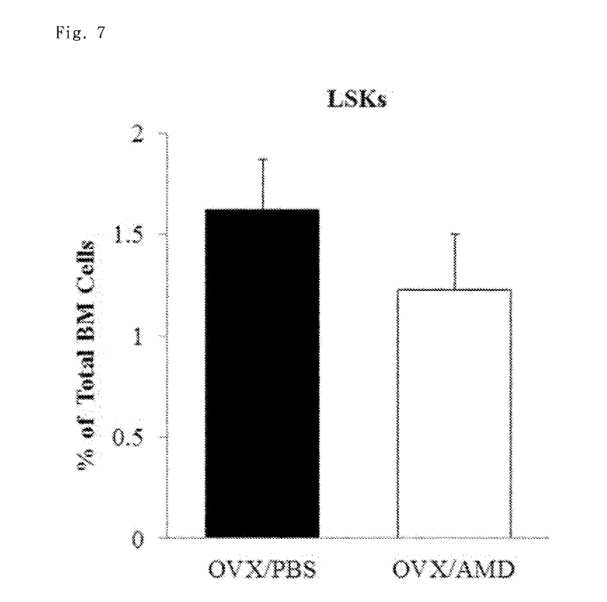

In order to determine whether AMD3100 may mobilize HSPCs from bone marrow in OVX mice, AMD3100 or PBS was administered to OVX mice for 21 days. FIG. 5 is a view illustrating the effect of AMD3100 on HSPC mobilization by CFU assay in blood. As illustrated in FIG. 5, the number of CFU cells showed a significant increase in AMD3100-treated OVX mice compared to PBS-treated OVX mice. Flow cytometric analysis was performed for evaluation of the percentage of HSPCs in bone marrow. The percentage of Lin⁻Sca-1⁺c-Kit⁺ (LSK) cells showed a decrease in AMD3100-treated OVX mice, compared with PBS-treated OVX mice in bone marrow, although this did not reach statistical significance (FIGS. 6 & 7). These data indicate that AMD3100 induces mobilization of HSPCs from bone marrow to blood in a model of OVX-induced osteoporosis.

### EXAMPLE 12

### Confirming Relief of OVX-Induced Bone Loss of AMD3100 Through Reduction of Osteoclast Deposition onto Bone Surfaces

In order to confirm the effect of AMD3100 on different bone parameters, micro-CT analysis was performed for the assessment of BMD, BMC, BVF, TMD, Tb.N., Tb.Sp., Cr.BMD. and Cr.BMC.

FIG. 8 is a view illustrating micro-CT images of the distal femur in each group. As illustrated in FIG. 8, increased bone density was observed in AMD3100-treated OVX mice compared with PBS-treated OVX mice.

FIG. 9 illustrates the BMD, BVF, BMC, TMD, Tb.N., Tb.Sp., Cr.BMD and Cr.BMC in the cortical bone and trabecular bone. As illustrated in FIG. 9, BMD showed an increase in AMD3100-treated OVX mice compared with PBS-treated OVX mice; however, BVF, BMC, TMD, Tb.N., Tb.Sp., Cr.BMD and Cr.BMC were similar between the groups.

Osteoporosis is likely the result of osteoclastic deposition rather than osteoblastic defects. Therefore, in order to confirm whether mobilization of AMD3100 affected osteoclast differentiation, first, it is confirmed whether HSPCs differentiate into mature functional osteoclasts by treatment with AMD3100.

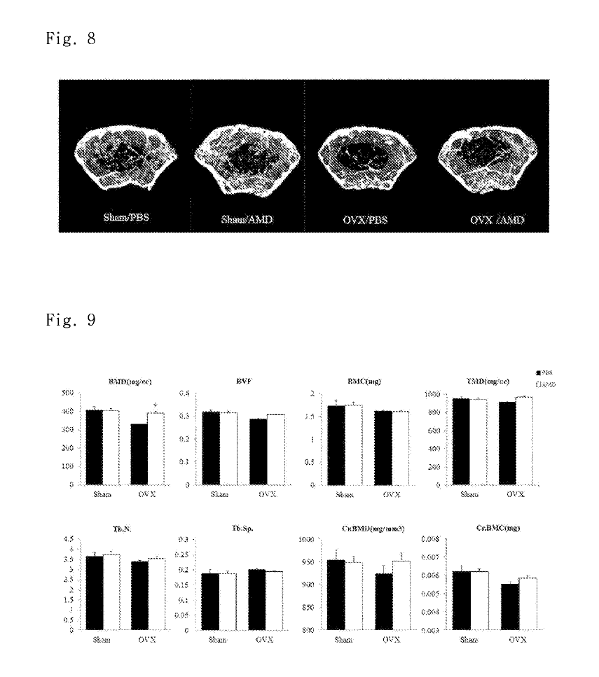

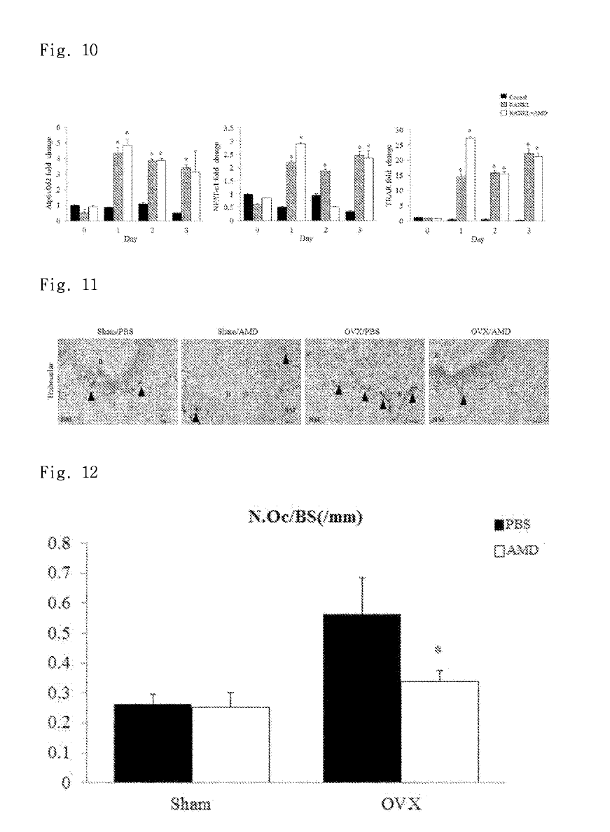

The results are illustrated in FIG. 10. As illustrated in FIG. 10, AMD3100 did not affect osteoclast differentiation.

Also, TRAP staining was performed for detection of osteoclasts in the trabecular region of OVX mice. FIG. 11 is a view illustrating the osteoclasts in the trabecular region of OVX mice. As illustrated in FIG. 11, the number and size of TRAP+active osteoclasts (black arrowhead) showed a decrease in the trabecular region in AMD3100-treated OVX mice.

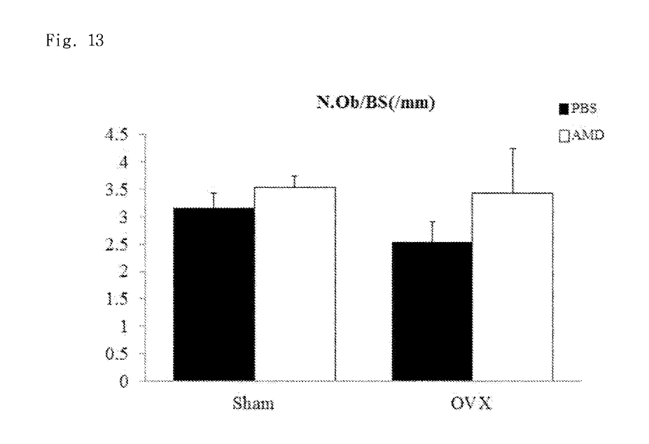

Histomorphometric analysis was performed for determination of the number of osteoclasts. FIG. 12 is a histogram illustrating the osteoclast number/bone surface [N.Oc/BS (/mm)]. As illustrated in FIG. 12, a decrease in the number of osteoclasts was observed in AMD3100-treated OVX mice compared with PBS-treated OVX mice. Also, H&E staining was performed in order to confirm the effect of AMD3100 on the number of osteoblasts.

The results are illustrated in FIG. 13. As illustrated in FIG. 13, osteoblast numbers did not change significantly among the AMD3100 treatment groups.

Taken together, the data demonstrate that treatment with AMD3100 induced mobilization of BM-derived HSPCs into blood, leading to amelioration of bone loss in a model of OVX-induced osteoporosis by reducing the number of osteoclasts attached to the bone surface.

What is claimed is:

1. A composition for preventing or treating bone diseases, comprising AMD3100 represented by the following formula 1 or a pharmaceutically acceptable salt thereof as an active ingredient:

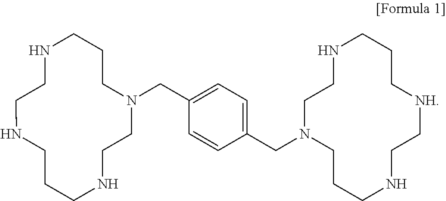 [SMILES: c1cc(CN2CCCNCCNCCCNCC2)ccc1CN1CCCNCCNCCCNCC1]

2. The composition of claim 1, wherein the bone disease is any one selected from the group consisting of osteoporosis, osteomalacia, rickets, fibrous osteitis, aplastic bone disorder, and metabolic bone disease.

3. The composition of claim 1, wherein the AMD3100 releases hematopoietic stem cell into blood stream, and reduces osteoclast within bone marrow.

4. The composition of claim 1, wherein the bone disease is osteoporosis.

5. A food composition for preventing or treating bone diseases, comprising AMD3100 represented by the following formula 1 as an active ingredient: 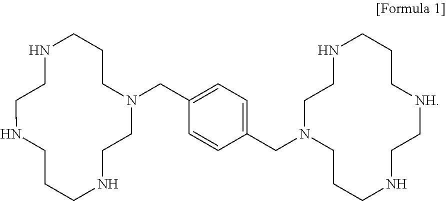 [SMILES: c1cc(CN2CCCNCCNCCCNCC2)ccc1CN1CCCNCCNCCCNCC1]
